(19) United States
(12) Patent Application Publication
(10) Pub. No.: US 2025/0332622 A1
(43) Pub. Date: Oct. 30, 2025

(54) STERILIZER CHAMBER CLEANING APPARATUS AND METHOD OF USING SAME

(71) Applicant: American Sterilizer Company, Mentor, OH (US)

(72) Inventors: Matthew Dailey, Bay Village, OH (US); Matthew M. Marchinetti, Fairview, PA (US); James D. Barrett, JR., Painesville, OH (US)

(21) Appl. No.: 19/193,219

(22) Filed: Apr. 29, 2025

Related U.S. Application Data
(60) Provisional application No. 63/640,530, filed on Apr. 30, 2024.

(51) Int. Cl. B08B 9/00; A61L 2/07; B08B 13/00

(52) U.S. Cl. CPC B08B 9/00; A61L 2/07; B08B 13/00; A61L 2202/17

(57) ABSTRACT

A compact and portable cleaning apparatus for cleaning surfaces of a chamber of a steam sterilizer and the like, and an associated method of using said apparatus to provide a thorough, convenient, and efficient cleaning process.

### FIELD OF THE INVENTION

The present invention relates generally to the field of chemical cleaning of surfaces, and more particularly to a compact and portable apparatus for cleaning surfaces of a chamber of a steam sterilizer and the like, and an associated method of using said apparatus to provide a thorough, convenient, and efficient cleaning process.

### BACKGROUND OF THE INVENTION

Steam sterilizers (also referred to as autoclaves) are widely used in healthcare and industrial applications to kill harmful decontaminants (e.g., bacteria, viruses, fungi, and spores) on articles that are placed inside the treatment chamber of the steam sterilizer. The articles inside the chamber are subjected to steam under pressure during an exposure phase of a decontamination process. The articles in the chamber are heated to an appropriate sterilization temperature for a given amount of time. The moisture in the steam efficiently transfers heat to the articles to destroy decontaminants on the articles. For example, the steam destroys the protein structure of bacteria and spores.

Steam sterilizers are available in a range of sizes that varies based on the capacity needed for the area where the steam sterilizer will be used. For example, in a dental office a small steam sterilizer may simply sit on the countertop where the equipment only needs to sterilize small packs of instruments. An immediate-use small steam sterilizer is typically needed near an operating room, and may only need to process 1-3 trays of instruments at a time. Most healthcare facilities, however, have medium or large steam sterilizers in their Sterile Processing Department (SPD), which can process 15-20 trays of instruments per sterilization cycle, or even up to 625 lbs. of instruments per sterilization cycle depending on size.

Over time, minerals and other contaminants from the sterilization process accumulate on the chamber walls from the incoming steam, causing discoloration, hinder sterilizer performance, and even contaminate sterilized articles. Accordingly, for proper maintenance of steam sterilizers there is a need for periodic cleaning of the treatment chamber. In addition to sterilizer manufacturer's written instructions for chamber cleaning, the Association of periOperative Registered Nurses (AORN), the Association for the Advancement of Medical Instrumentation (AAMI), and the National Integrated Accreditation for Healthcare Organizations (NIAHO) have adopted policies and procedures for routine cleaning of sterilizer chambers in order to ensure acceptable levels of safety and quality in connection with the sterilization of articles. Manufacturers of steam sterilizers generally advise that chambers should be professionally cleaned at least once a year. However, in accordance with guidance from the AORN and AAMI, semi-annual or quarterly professional cleanings may be more appropriate depending upon usage volume and the quality of incoming steam.

A professional chamber cleaning typically includes: a manual prep chamber scrubbing, and a cleaning process where alkaline and acidic cleaning solutions are applied to the interior surfaces of the chamber using a chamber cleaning apparatus. A professional sterilizer chamber cleaning results in the removal of most substances built up on chamber walls, improved drying and heat transfer within chamber walls, reduced contamination, and casier routine operator cleaning.

Existing conventional chamber cleaning apparatus takes the form of a large, bulky and heavy apparatus that is difficult to transport and too large to be located inside of many sterilizer chambers. Therefore, the cleaning process is hindered by the dimensions and configuration of the existing chamber cleaning apparatus. U.S. Pat. Nos. 6,550,487 and 6,770,150 disclose an example of a typical existing chamber cleaning apparatus. Such cleaning apparatus is comprised of a large cart; a first cleaning fluid reservoir, which holds a first cleaning fluid for removing organic residues and passivating the chamber; a second cleaning fluid reservoir, which holds a second cleaning fluid for removing inorganic residues and passivating the chamber; a nozzle for spraying the cleaning fluid into the chamber; a first fluid supply line for fluidly connecting the first and second cleaning fluid reservoirs of cleaning fluid with the nozzle; a first pump that is fluidly connectable with the nozzle and the first and second cleaning fluid reservoirs for pumping the cleaning fluid to the nozzle; and a second pump that is fluidly connectable with a chamber outlet fitting for pumping the cleaning fluid out from the chamber. The cleaning apparatus is powered by a corded electrical connection with the mains power outlet.

In a typical cleaning operation, the nozzle is inserted through an access port of the sterilizer chamber door in order spray cleaning solution inside the interior of the chamber. Some sterilizers do not have a chamber door with an access port. In such cases, the sterilizer chamber door is removed and replaced with a temporary adapter door that has an access port dimensioned to receive the nozzle. After the cleaning process is completed, the temporary adapter door is removed and replaced with the original chamber door. Where a temporary adapter door is used, the interior side of the original chamber door is typically cleaned by a separate, convention cleaning method, such as a hand cleaning process.

In view of the foregoing, it can be appreciated that there are many shortcoming with existing chamber cleaning apparatus. As noted above, the existing chamber cleaning apparatus requires a large, heavy, and bulky cart to house the components of the cleaning apparatus. This cart imposes many ergonomic and space challenges. For instance, it is not possible to clean the interior side of sterilizer chamber doors that do not have an access port, since the entire cleaning apparatus cannot be located inside of the sterilizer chamber. In situations where a temporary adapter door is used, the chamber door is not cleaned by the chamber cleaning apparatus, but rather, a separate, manual cleaning process is needed in order to clean the interior surface of the chamber door. Furthermore, the power requirements for the existing cleaning apparatus requires the use of a corded connection to a mains power outlet.

The present invention provides a compact, portable, and self-contained apparatus for cleaning surfaces of a chamber of a steam sterilizer, and an associated method of using said apparatus.

### SUMMARY OF THE INVENTION

According to one aspect of the invention, a cleaning apparatus for cleaning a chamber includes: a reservoir for storing a cleaning chemistry; a spray nozzle for distributing the cleaning chemistry within the chamber: a fluid transport system for conveying the cleaning chemistry from the reservoir to the spray nozzle and returning the cleaning chemistry from the chamber to a reservoir; a power source for powering the pumping system; and a control unit for controlling operation of the pumping system.

In one embodiment, the control unit includes a start switch for initiating a cleaning cycle.

In one embodiment, the fluid transport system includes: a first pump for conveying the cleaning chemistry from the reservoir to the spray nozzle; and a second pump for returning the cleaning chemistry from the chamber to the reservoir.

In one embodiment, the reservoir includes a floor and a first port disposed in the floor, and the second pump comprises a suction port fluidically coupled to the first port.

In one embodiment, the reservoir includes a floor and at least one side wall, and the cleaning apparatus further includes: a second port disposed in the at least one side wall; and a selector valve having an output port and at least two input ports, the selector valve configured to selectively couple one of the at least two input ports to the output port, wherein the second pump is coupled to the output port of the selector valve, one of the at least two input ports is coupled to the first port, and the other of the at least two ports is coupled to the second port.

In one embodiment, the cleaning apparatus includes a housing having an interior chamber, wherein the second pump is configured to create a suction that draws fluid into the interior chamber.

In one embodiment, the reservoir comprises a floor and at least one side wall, and the cleaning apparatus further includes a first port disposed in the floor of the reservoir and a second port disposed in the at least one side wall of the reservoir, wherein the second pump includes a suction port fluidically coupled to the first port and an exhaust port fluidically coupled to the second port.

In one embodiment, the cleaning apparatus includes: a third port disposed in the at least one side wall; and a selector valve having an output port and at least two input ports, the selector valve configured to selectively couple one of the at least two input ports to the output port, wherein the suction port of the second pump is coupled to the output port of the selector valve, one of the at least two input ports is coupled to the first port, and the other of the at least two ports is coupled to the third port.

In one embodiment, the control unit includes: a first timer for activating the first pump; and a second timer for activating the second pump.

In one embodiment, the first and second pumps are selected from one of a rotary vane pump and a bilge pump.

In one embodiment, the spray nozzle is rotatable.

In one embodiment, the power source includes a rechargeable battery.

In one embodiment, the cleaning chemistry includes a descaler and neutralizing agent.

In one embodiment, the cleaning chemistry includes an acid-based cleaning agent.

In one embodiment, the reservoir is at least a portion of an interior chamber of a housing that houses the first and second pumps, the power source, and the control unit.

In one embodiment, the cleaning apparatus includes a housing having an interior to house the fluid transport system, the reservoir, the power source, and the control unit.

In one embodiment, the control unit includes a wireless remote control.

According to another aspect of the invention, a method of cleaning a chamber of a steam sterilizer using a cleaning apparatus is provided, the method including: opening the door of a steam sterilizer to gain access to the sterilizer chamber; locating the cleaning apparatus within a chamber of the steam sterilizer, said cleaning apparatus adapted to spray a cleaning chemistry inside the chamber; initiating operation of the cleaning apparatus to commence a cleaning cycle; closing the door of the steam sterilizer to enclose the cleaning apparatus within the sterilizer chamber; reopening the door after completion of the cleaning cycle; and removing the cleaning apparatus from the chamber of the steam sterilizer.

In one embodiment, operation of the cleaning cycle is initiated by use of a start switch.

In one embodiment, operation of the cleaning cycle is initiated by a wireless remote control.

In one embodiment, the cleaning apparatus is fully self-contained within the sterilizer chamber.

In one embodiment, initiating operation of the cleaning apparatus includes creating a suction within a housing of the cleaning apparatus.

According to another aspect of the invention, a method for simultaneously cleaning a plurality of steam sterilizer chambers using a plurality of cleaning apparatus is provided, the method including: opening the doors of a plurality of steam sterilizers to gain access to each of respective sterilizer chambers; locating a cleaning apparatus within each sterilizer chamber of the steam sterilizers, said cleaning apparatus adapted to spray a cleaning chemistry inside the sterilizer chamber; initiating operation of each of the cleaning apparatus to commence a cleaning cycle; closing the door of each steam sterilizer to enclose each cleaning apparatus within each of the sterilizer chambers; reopening the doors of each steam sterilizer after completion of the cleaning cycle; and removing each of the cleaning apparatus from the sterilizer chambers.

In one embodiment, operation of the cleaning cycle for at least one of the cleaning apparatus is initiated by use of a start switch.

In one embodiment, operation of the cleaning cycle is initiated for at least one of the cleaning apparatus by use of a wireless remote control.

In one embodiment, each of said cleaning apparatus are respectively fully self-contained within the sterilizer chambers.

In one embodiment, initiating operation of each cleaning apparatus includes creating suction within a housing of each cleaning apparatus.

These and other advantages will become apparent from the following description of illustrated embodiments taken together with the accompanying drawings and the appended claims.

### BRIEF DESCRIPTION OF THE DRAWINGS

The invention may take physical form in certain parts and arrangement of parts, an embodiment of which will be described in detail in the specification and illustrated in the accompanying drawings which form a part hereof, and wherein:

FIG. 1 is a schematic block diagram of the sterilizer chamber cleaning apparatus, according to an embodiment of the present invention; and

FIG. 2 is a perspective view of an embodiment of the housing of the sterilizer chamber cleaning apparatus shown in FIG. 1;

FIG. 3 is an exploded view of the housing shown in FIG. 2;

FIG. 4 is a block diagram of a conventional steam sterilizer; and

FIG. 5 is a schematic block diagram of the sterilizer chamber cleaning apparatus, according to a second embodiment of the present invention.

FIG. 6 is a schematic block diagram of the sterilizer chamber cleaning apparatus, according to a third embodiment of the present invention.

### DETAILED DESCRIPTION OF THE INVENTION

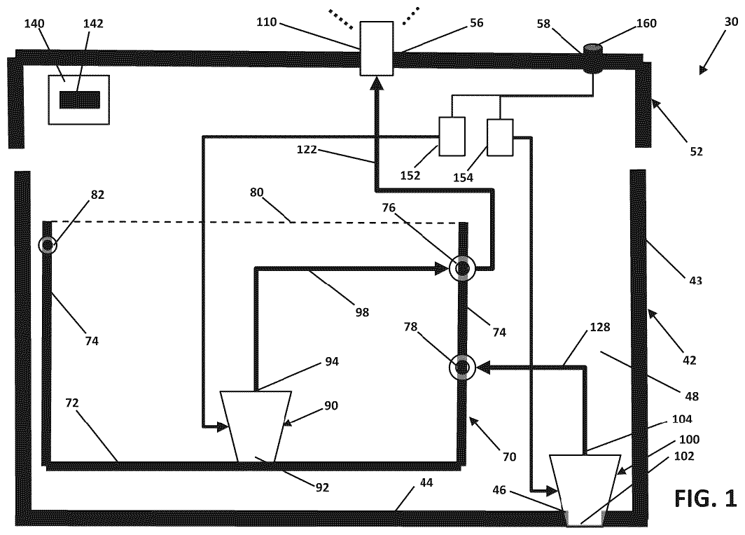

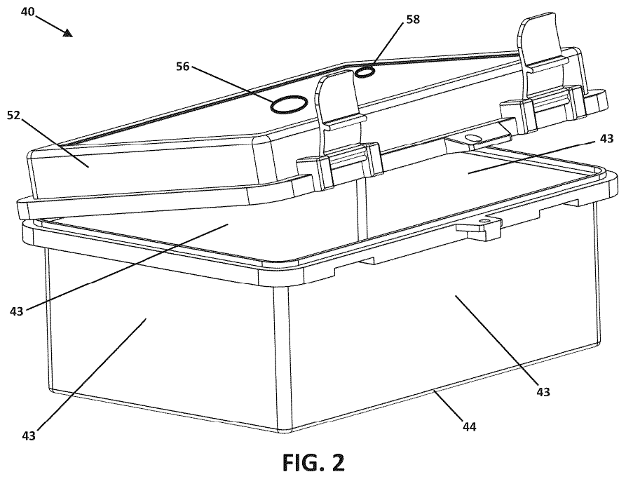

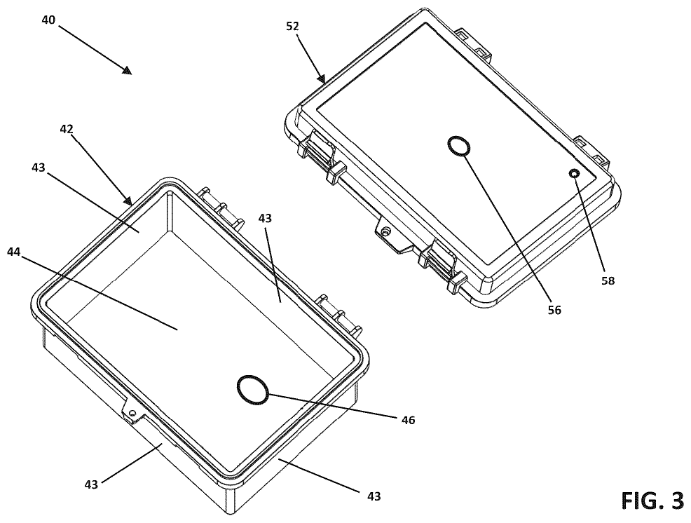

Referring now to the drawings wherein the showings are for the purposes of illustrating an embodiment of the invention only and not for the purposes of limiting same, FIG. 1 shows a schematic block diagram of a sterilizer chamber cleaning apparatus 30, according to one embodiment of the present invention. Cleaning apparatus 30 is generally comprised of a housing 40 (shown in detail in FIGS. 2 and 3), a tank or reservoir 70, a spray pump 90, a return pump 100, a rotating spray nozzle 110, a nozzle fluid supply line 122, a fluid return line 128, a power system 140, and a control unit including first and second timers 152, 154, a start switch 160, and an electromagnetic relay switch (not shown).

Referring now to FIGS. 2 and 3, housing 40 according to an embodiment of the present invention will be described in detail. It should be understood that housing 40 may take a wide variety of alternative forms, and the embodiment shown in FIGS. 2 and 3 is provided solely for purposes of illustrating one embodiment of the present invention, and not for limiting same. Housing 40 functions to provide a lightweight, compact, waterproof, and ergonomic case to house the components of cleaning apparatus 30, which are described in detail below. For example, housing 40 may take the form an SKB iSeries Injection Molded Mil-Standard Waterproof Case, model no. 31-1510-9B-E SKB. This example case is molded of ultra high-strength polypropylene copolymer resin, featuring a gasket-sealed, water and dust tight, submersible design that is resistant to corrosion and impact damage.

In the embodiment of housing 40 shown in FIGS. 2 and 3, housing 40 is generally comprised of a base portion 42 and a lid portion 52 that is connected to base portion 42 by a hinge member. Base portion 42 is comprised of side walls 43 and a bottom wall 44 (also referred to as a floor). Side walls 43 and bottom wall 44 define a housing interior chamber 48. A bottom port 46 (FIG. 3) is formed in bottom wall 44 for return pump 100, which is described below. As indicated above, lid portion 52 is connected to base portion 42 by the hinge member, thereby allowing convenient access to the interior of housing 40. First and second openings 56 and 58 are formed in lid portion 52. First opening 56 is dimensioned to receive spray nozzle 110, and second opening 58 is dimensioned to provide access to a start switch 160. In the illustrated embodiment, the housing 40 has dimensions similar to a large tackle or tool utility box with an interior volume in the range of approximately 30-40 L.

Returning now to FIG. 1, reservoir 70 is comprised of a bottom wall 72, side wall 74, and a removable lid 80 for enclosing the interior of reservoir 70. An outlet port 76 and an inlet port 78 are formed in side wall 74. A vent overflow outlet 82 is provided proximate to the upper edge of side wall 74. Furthermore, an opening for receiving electrical wires (not shown) is also be formed in side wall 74 or lid 80 of reservoir 70. Reservoir 70 contains a liquid cleaning chemistry. In the illustrated embodiment, reservoir 70 has an interior volume of approximately 5-15 L. According to an alternative embodiment of the present invention, housing interior chamber 48, defined by side walls 43 and bottom wall 44 of housing 40, may serve as a reservoir that replaces reservoir 70.

Spray pump 90 is located in the interior of reservoir 70, and includes an inlet 92 for receiving fluid located inside reservoir 70 and an outlet 94 for discharging fluid from spray pump 90. It should be appreciated that inlet 92 may be connected to tubing (not shown) which receives the fluid from reservoir 70. An outlet line 98 fluidly connects outlet 94 of spray pump 90 to outlet port 76 of reservoir 70. A supply line 122 fluidly connects outlet port 76 to spray nozzle 110. Activation of spray pump 90 supplies fluid from reservoir 70 to spray nozzle 110.

Return pump 100 includes an inlet 102 for receiving fluid through bottom port 46 of housing 40 and an outlet 104 for discharging fluid from the pump. The fluid received through bottom port 46 is fluid recovered from the floor of the chamber of the sterilizer undergoing a cleaning operation. A return line 128 fluidly connects outlet 104 of return pump 100 with inlet port 78 of reservoir 70. Accordingly, activation of return pump 100 removes fluid from the floor of the sterilizer chamber and returns it to reservoir 70 for re-use. It should be appreciated that inlet 102 may be connected to tubing (not shown) which receives the fluid recovered from the floor of the sterilizer chamber.

Spray pumps 90 and return pump 100 collectively form a fluid transport system for cleaning apparatus 30. In the illustrated embodiment of the present invention, spray pump 90 and return pump 100 may take the form of a variety of suitable compact pumps, including, but not limited to a rotary vane pump or a bilge pump. Some example bilge pumps include Amarine automatic submersible bilge pump, model no. AMBP1-G1100-1, and Jabsco 18660-0121 Water Puppy bilge pump. The Jabsco bilge pump is a flexible impeller pump having a neoprene seal, ½ inch NPT and 1 inch hose barb ports, coupled to a 12V DC motor. The Jabsco bilge pump is self-priming and has 6.3 GPM (24 LPM) flow,

Rotating spray nozzle 110 may take the form of a variety of suitable nozzles that can provide a 360 degree spray pattern. An example of a suitable nozzle is the DERNORD stainless steel rotary spray ball (Part No. DNCA1501149) having a ball diameter of 38 mm, a working pressure of 1-2 bar, and a cleaning radius of 0.5-1.5 m. Another suitable nozzle is the Hydro Whirl® Stinger (HWS2) rotating tank cleaning nozzle from BETE Fog Nozzles, Inc. In the illustrated embodiment, spray nozzle 110 is mounted in first opening 56 of lid portion 52, such that the spray ball of spray nozzle 110 is located on the exterior of housing 40, as depicted in FIG. 1.

In the illustrated embodiment, cleaning apparatus 30 also includes a float switch (not shown) that is located in the interior of reservoir 40. The float switch prevents an overflow condition in reservoir 70.

Power system 140 is comprised of a battery 142 removably mountable to a battery receptacle (not shown). In the illustrated embodiment, battery 142 takes the form of a conventional 20V 7AH rechargeable lithium-ion battery. Power system 140 provides electrical power to spray pump 90 and return pump 100.

The control unit provides system control of cleaning apparatus 30. The control unit is comprised of first timer 152, second timer 154, a start switch 160, and one or more electromagnetic relay switches (not shown), which is electrically connected to timers 152, 154 and pumps 90, 100. In one embodiment, the control unit may also include a conventional microcontroller, wherein the first and second timers may be implemented through code executed by the controller. Start switch 160 is electrically connected with timers 152 and 154 to initiate a cleaning cycle by activating first and second timers 152, 154. First timer 152 is electrically connected with spray pump 90, and second timer 154 is electrically connected with return pump 100, via the one or more electromagnetic relay switches.

First timer 152 is a conventional timer for activation of spray pump 90, and second timer 154 is a conventional timer for activation of return pump 100. Operation of timers 152, 154 will be described below. Start switch 160 is electrically connected to first and second timers 152 and 154. In the illustrated embodiment, start switch 160 is mounted in second opening 58 of lid portion 52, such that user access to start switch 160 is provided on the exterior of housing 40, as depicted in FIG. 1. Depressing start switch 160 activates first and second timers 152, 154, which in turn, activate spray pump 70 and return pump 100 (via the one or more electromagnetic relay switches) for commencing a cleaning cycle, as will be explained below.

In accordance with an illustrated embodiment of the present invention, the liquid cleaning chemistry takes the form of an acid-based cleaning agent, such as ProKlenz® RESTORE from STERIS Corporation. ProKlenz® RESTORE is high performance acid-based cleaner, low foaming acid based product for type I rouge removal and all-purpose cleaning. This acid-based cleaning agent is a mixture comprised of 1-Hydroxyethane-1,1-diphosphonic acid, oxalic acid, 1-Amino-2-propanol. The liquid cleaning chemistry may also take the form of a descaler and neutralizing agent, such as Prolystica® RESTORE Descaler and Neutralizing Agent from STERIS Corporation. Prolystica® RESTORE Descaler and Neutralizing Agent is a low-foaming acid-based formula that effectively neutralizes high-alkaline detergent residue. This cleaning agent is comprised of a mixture of citric acid and oxalic acid.

In accordance with the illustrated embodiment of the present invention, cleaning apparatus 30 has an empty dry weight of about 16 lbs.

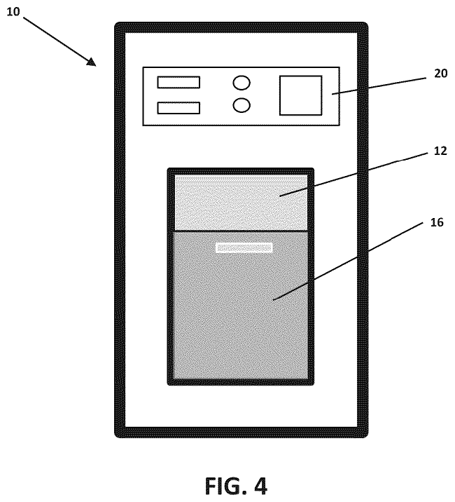

Cleaning apparatus 30 is adapted for cleaning the sterilization chamber of a conventional steam sterilizer, such as steam sterilizer 10 schematically illustrated in FIG. 4. Steam sterilizer 10 includes a chamber 12, and a sliding door 16 that is movable between an open or downward position and a closed or upward position. Door 16 provides access to chamber 12 in the open position and seals chamber 12 in the closed position. Some typical steam sterilizers from AMSCO® and Getinge® have sterilizer chambers with interior volumes ranging from about 100 L-1000 L.

It should be appreciated that while the illustrated embodiment of the present invention has a pumping system that is comprised of two (2) separate pumps (i.e., spray pump 90 and return pump 100), which are simultaneously or individually operable, it is contemplated that the pumping system may be modified to replace the two (2) pumps with a single pump that has a 3-way flow control valve having an actuator for controlling the direction of fluid flow according to a required task. In this manner, the single pump would be operable to both spray cleaning chemistry into the sterilizer chamber and to suck up fluid from the floor of the sterilizer chamber.

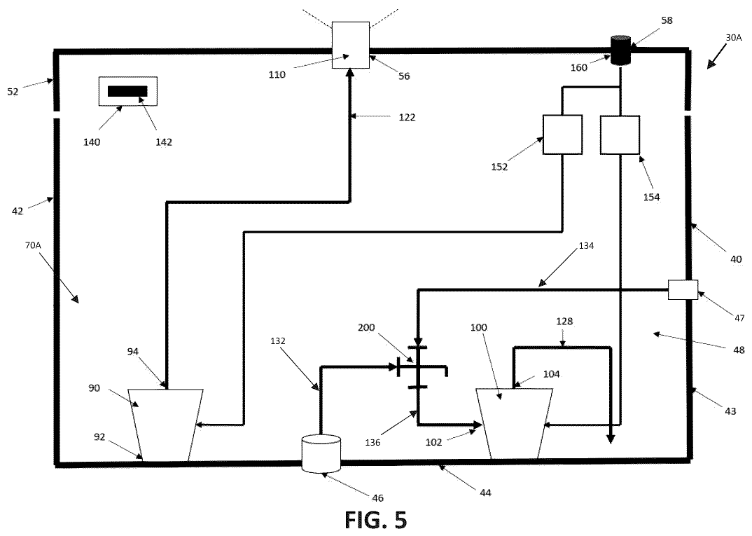

Referring now to FIG. 5, there is shown a sterilizer chamber cleaning apparatus 30A according to a second embodiment of the present invention. In this embodiment, reservoir 70 of cleaning apparatus is omitted and at least a portion of housing interior chamber 48 serves as a reservoir 70A. In addition, cleaning apparatus 30A includes a three-way valve 200 and a side port 47 formed in side wall 43. Three-way valve 200 is fluidly connected with (i) bottom port 46 in bottom wall 44 via a first recycle line 132, (ii) side port 47 via a second recycle line 134, and (iii) inlet 102 of return pump 100 via an inlet line 136. It should be appreciated that side port 47 can be used for fluid connection of return pump 100 to a drain adapter (not shown) associated with a drain located inside chamber 12 of sterilizer 10. In this regard, a suction line (not shown) is connected between side port 47 and the drain adapter. In the illustrated embodiment, three-way valve 200 is a manually-controlled valve that is controlled by the user to select the fluid connection of inlet line 136 to either bottom port 46 (via recycle line 132) or side port 47 (via recycle line 134). It is also contemplated that three-way valve 200 may take the form of an automatic valve that is controlled by the control unit.

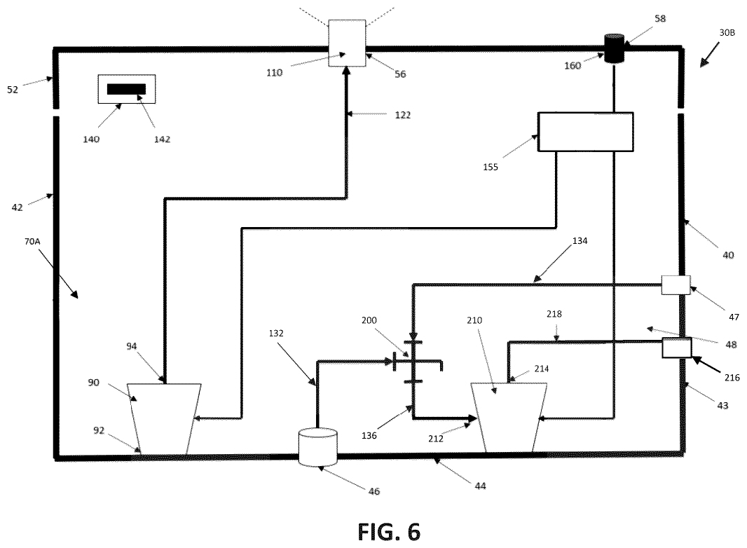

Referring now to FIG. 6, there is shown a sterilizer chamber cleaning apparatus 30B according to a third embodiment of the present invention. In this embodiment, and like the second embodiment illustrated in FIG. 5, reservoir 70 of cleaning apparatus is omitted and at least a portion of housing interior chamber 48 serves as a reservoir 70A. In addition, the return pump 100 is replaced with a negative pressure pump 210. In the embodiment of FIG. 6, spray pump 90 and negative pressure pump 210 collectively form a fluid transport system for cleaning apparatus 30B.

As used herein, a negative pressure pump is a device that generates a pressure lower than atmospheric pressure. Negative pressure pumps generally employ a diaphragm, piston mechanism or blower to generate negative pressure, although other means for generating negative pressure fall within the scope of the invention. In one embodiment, the negative pressure pump 210 may be embodied as a vacuum blower configured to move air or gas from one location to another location. Vacuum blowers operate by drawing in air and then pushing the air out at a higher pressure.

In the embodiments that follow, the invention is described in the context of a negative pressure pump in the form of a vacuum blower. It should be appreciated, however, that other types of negative pressure pumps may be employed, and these other types of negative pressure pumps fall within the scope of the invention.

The vacuum blower 210 includes an inlet (suction) port 212 and an outlet (exhaust) port 214. The outlet (exhaust) port 214 of the vacuum blower 210 is fluidically coupled to exhaust port 216 via exhaust line 218 to exhaust air out of the chamber 48. Like the embodiment of FIG. 5, cleaning apparatus 30B includes three-way valve 200, bottom port 46 and side port 47. Three-way valve 200 is fluidly connected with (i) bottom port 46 in bottom wall 44 via a first recycle line 132, (ii) side port 47 via a second recycle line 134, and (iii) inlet (suction) 212 of vacuum blower 210 via an inlet line 136. It should be appreciated that like the embodiment of FIG. 5, side port 47 can be used for fluid connection to a drain adapter (not shown) associated with a drain located inside chamber 12 of sterilizer 10.

The three-way valve 200 can be manually (user) or automatically (microcontroller) controlled to selectively couple the suction port 212 of the vacuum blower 210 to one of the bottom inlet port 47 or the side port 47. The vacuum blower 210 is operative to create a negative pressure inside the enclosure, and this negative pressure draws fluid back into the reservoir 70A through bottom port 46. A single microcontroller 155 replaces the two microcontrollers of the first and second embodiments, the single microcontroller 155 operatively coupled to the spray pump 90, the vacuum blower 210 and optionally to the selector valve 220 to control operation thereof.

Operation and method of use of cleaning apparatus 30 according to one embodiment of the present invention will now be described with reference to the cleaning of chamber 12 of sterilizer 10 shown in FIG. 4. First, lid portion 52 of housing 40 is opened to provide access to the interior of housing 40. Lid 80 of reservoir 70 is then removed and reservoir 70 is filled with a liquid cleaning chemistry. Lid 80 is returned to enclose the interior of reservoir 70 and lid portion 52 of housing 40 is returned to the closed position.

Next, the user moves door 16 to the open position, and locates cleaning apparatus 30 inside chamber 12 such that cleaning apparatus 30 is placed flush on the floor of chamber 12. It should be appreciated that cleaning apparatus 30 is fully self-contained within the sterilizer chamber.

Next, the user initiates a cleaning cycle by depressing start switch 160.

Thereafter, the user moves door 16 to the closed position. First timer 152 is preprogrammed with a startup time delay (e.g., 15-30 seconds). Accordingly, first timer 152 begins the startup delay immediately following depression of the start switch 160. The purpose of the startup delay time is to provide the user with sufficient time to move door 16 to the closed position.

After the startup time delay has elapsed, first timer 152 activates spray pump 90, via the one or more electromagnetic relay switches, to initiate a cleaning cycle. First timer 152 is preprogrammed with a cleaning time (e.g., 30 minutes) during which spray pump 90 is continuously activated.

Second timer 154 is programmed to activate return pump 100, via the one or more electromagnetic relay switches, following a programmed time delay (e.g., 10 seconds) after spray pump 90 is activated by first timer 152. During the 30 minute cleaning cycle, return pump 100 is controlled by the float switch to prevent an overflow condition.

First timer 152 deactivates spray pump 90 at the expiration of the 30 minute cleaning cycle. Second timer 154 deactivates the return pump 100 a short time (e.g., a few seconds) after spray pump 90 is deactivated so that return pump 100 can suction up fluid remaining on the floor of the sterilizer chamber. This prevents spillage of cleaning chemistry when the chamber door is opened by service personnel to remove cleaning apparatus 30, following completion of a cleaning cycle.

Operation of cleaning apparatus 30A according to the second embodiment of the present invention will now be described with reference to the cleaning of chamber 12 of sterilizer 10 shown in FIG. 4. First, lid portion 52 of housing 40 is opened to provide access to interior chamber 48 of housing 40. As indicated above, at least a portion of interior chamber 48 serves as reservoir 70A. Next, reservoir 70A is filled to an appropriate volume of a liquid cleaning chemistry. The user may configure manually-controlled three-way valve 200 to select the desired suction from either bottom port 46 (via first recycle line 132) or side port 47 (via second recycle line 134). Thereafter, lid portion 52 of housing 40 is returned to the closed position.

Next, the user moves door 16 of sterilizer 10 to the open position, and locates cleaning apparatus 30A inside chamber 12 such that cleaning apparatus 30A is placed flush on the floor of chamber 12. Cleaning apparatus 30A is fully self-contained within the sterilizer chamber.

Next, the user initiates a cleaning cycle by depressing start switch 160. Thereafter, the user moves door 16 of sterilizer 10 to the closed position. As discussed above, first timer 152 is preprogrammed with a startup time delay (e.g., 15-30 seconds). Accordingly, first timer 152 begins the startup delay immediately following depression of the start switch 160.

After the startup time delay has elapsed, first timer 152 activates spray pump 90, via the one or more electromagnetic relay switches, to initiate a cleaning cycle. As mentioned above, first timer 152 is preprogrammed with a cleaning time (e.g., 30 minutes) during which spray pump 90 is continuously activated.

Second timer 154 is programmed to activate return pump 100, via the one or more electromagnetic relay switches, following a programmed time delay (e.g., 10 seconds) after spray pump 90 is activated by first timer 152. Return pump 100 recycles fluid recovered from chamber 12 through bottom port 46 (via first recycle line 132) or through side port 47 (via second recycle line 134), depending upon the configuration of three-way valve 200. For example, when three-way valve 200 is configured for suction through bottom port 46, recycled fluid is received by return pump 100 through bottom port 46, via first recycle line 132, three-way valve 200, and inlet line 136. The recycled fluid is discharged back into reservoir 70A by return pump 100 via return line 128.

First timer 152 deactivates spray pump 90 at the expiration of the 30 minute cleaning cycle. Second timer 154 deactivates the return pump 100 a short time (e.g., a few seconds) after spray pump 90 is deactivated so that return pump 100 can suction up fluid remaining on the floor of sterilizer chamber 12.

Operation of cleaning apparatus 30B according to the third embodiment of the present invention will now be described with reference to the cleaning of chamber 12 of sterilizer 10 shown in FIG. 4. First, lid portion 52 of housing 40 is opened to provide access to interior chamber 48 of housing 40. As indicated above, at least a portion of interior chamber 48 serves as reservoir 70A. Next, reservoir 70A is filled to an appropriate volume of a liquid cleaning chemistry. At this time the operator may position three-way valve 200 in the desired position for fluid recovery (e.g., bottom port 46 or side port 47).

Next, the user moves door 16 of sterilizer 10 to the open position, and locates cleaning apparatus 30B inside chamber 12 such that cleaning apparatus 30B is placed flush on the floor of chamber 12. Cleaning apparatus 30B is fully self-contained within the sterilizer chamber.

Next, the user initiates a cleaning cycle by depressing start switch 160. Thereafter, the user moves door 16 of sterilizer 10 to the closed position. Immediately following depression of the start switch 160, microcontroller 155 initiates a first timer (e.g., a first internal software timer) that is preprogrammed with a startup time delay (e.g., 15-30 seconds) and a cleaning time (e.g., 30 minutes). During the startup time delay, the microcontroller 155 may cause an audible and/or visual alert to be generated to provide confirmation that a cleaning cycle has been initiated and that the cleaning cycle will commence upon expiration of the startup time delay. After the startup time delay has elapsed, microcontroller 155 activates spray pump 90 and vacuum blower 210, via the one or more electromagnetic relay switches, to initiate a cleaning cycle.

During the cleaning cycle, the microcontroller 155 may operate the spray pump 90 in an on-off cycle, e.g., 30 seconds on and 30 seconds off. Such on/off cycling may be useful in preventing the reservoir 70A from becoming depleted. The microcontroller 155 may operate the vacuum blower 210 continuously during the cleaning cycle so as to maximize recovery of the cleaning fluid back into the reservoir and reduce the likelihood of the reservoir becoming depleted.

Outlet port 214 of vacuum blower 210 evacuates through exhaust port 216 (via exhaust line 218). Depending on the position of the three-way valve 200, the vacuum blower 210 creates suction at bottom port 46 (via recycle lines 132, 136) or at side port 47 (via recycle lines 134, 136). The suction created at the bottom port 46 or at the side 47 recycles fluid recovered from chamber 12 through the respective port and back into the reservoir 70A.

Upon the expiration of the 30-minute cleaning cycle as monitored by the first timer, the microcontroller 155 deactivates spray pump 90. A few seconds after the spray pump 90 is deactivated, e.g., 20 seconds, the microcontroller 155 deactivates the vacuum blower 210. The delay in deactivating the vacuum blower 210 relative to the spray pump 90 enables recovery of cleaning fluid that may still be draining down the walls of the chamber 12. At the end of the cleaning cycle the microcontroller 155 may cause an audible and/or visual alert to be generated to indicate the cleaning cycle is complete.

In the event the charge stored in battery 142 drops below a prescribed level, the microcontroller 155 may stop the spray pump 90. However, the microcontroller 155 may continue to operate the vacuum blower 210 for a preset time period so as to recover fluid within the chamber 12 before the battery 142 becomes fully discharged. The microcontroller 155 may cause an audible and/or visual alert to be generated to indicate the battery is low.

In addition to operation of the vacuum blower 210 in automatic mode as described above, it also me be operated in a manual mode. In manual mode, the vacuum blower 210 may be activated to enable an operator to manually recover fluid.

It is contemplated that the cleaning apparatus according to the present invention can be modified in a variety of ways. For example, the cleaning apparatus may be scaled up for larger sterilizer chambers. In addition, start switch 160 of the control unit may be replaced by a conventional wireless remote control to allow a cleaning cycle of the cleaning apparatus to be remotely initiated by service personnel.

Moreover, it is contemplated that multiple cleaning apparatus according to the present invention can be deployed simultaneously in a plurality of sterilizer chambers to simultaneously clean multiple sterilizer chambers. Accordingly, multiple sterilizer chambers can be cleaned in parallel using multiple cleaning apparatus. It should be appreciated that a conventional wireless remote control may be implemented to simultaneously initiate cleaning cycles for a plurality of cleaning apparatus. An exemplary method of operation includes the following steps for simultaneously cleaning a plurality of steam sterilizer chambers using a plurality of cleaning apparatus: opening the doors of a plurality of steam sterilizers to gain access to each of respective sterilizer chambers; locating a cleaning apparatus within each sterilizer chamber of the steam sterilizers, said cleaning apparatus adapted to spray a cleaning chemistry inside the sterilizer chamber; initiating operation of each of the cleaning apparatus to commence a cleaning cycle; closing the door of each steam sterilizer to enclose each cleaning apparatus within each of the sterilizer chambers; reopening the doors of each steam sterilizer after completion of the cleaning cycle; and removing each of the cleaning apparatus from the sterilizer chambers.

### Exemplary Aspects

The following exemplary aspects are provided, the numbering of which is not to be construed as designating levels of importance:

Aspect 1 provides a cleaning apparatus for cleaning a chamber, comprising:
a reservoir for storing a cleaning chemistry;
a spray nozzle for distributing the cleaning chemistry within the chamber:
a fluid transport system for conveying the cleaning chemistry from the reservoir to the spray nozzle and returning the cleaning chemistry from the chamber to a reservoir;
a power source for powering the pumping system; and
a control unit for controlling operation of the pumping system.

Aspect 2 provides a cleaning apparatus of Aspect 1, wherein the control unit includes: a start switch for initiating a cleaning cycle.

Aspect 3 provides a cleaning apparatus of Aspects 1-2, wherein the fluid transport system is comprised of:
a first pump for conveying the cleaning chemistry from the reservoir to the spray nozzle; and
a second pump for returning the cleaning chemistry from the chamber to the reservoir.

Aspect 4 provides a cleaning apparatus of Aspect 3, wherein the reservoir comprises a floor and a first port disposed in the floor, and the second pump comprises a suction port fluidically coupled to the first port.

Aspect 5 provides a cleaning apparatus of Aspect 3, wherein the reservoir comprises a floor and at least one side wall, further comprising:
a second port disposed in the at least one side wall; and
a selector valve having an output port and at least two input ports, the selector valve configured to selectively couple one of the at least two input ports to the output port,
wherein the second pump is coupled to the output port of the selector valve, one of the at least two input ports is coupled to the first port, and the other of the at least two ports is coupled to the second port.

Aspect 6 provides a cleaning apparatus of Aspects 3-5, further comprising a housing having an interior chamber, wherein the second pump is configured to create a suction that draws fluid into the interior chamber.

Aspect 7 provides a cleaning apparatus of Aspects 3-6, wherein the reservoir comprises a floor and at least one side wall, further comprising a first port disposed in the floor of the reservoir and a second port disposed in the at least one side wall of the reservoir, wherein the second pump comprises a suction port fluidically coupled to the first port and an exhaust port fluidically coupled to the second port.

Aspect 8 provides a cleaning apparatus of Aspects 3-7, further comprising:
a third port disposed in the at least one side wall; and
a selector valve having an output port and at least two input ports, the selector valve configured to selectively couple one of the at least two input ports to the output port,
wherein the suction port of the second pump is coupled to the output port of the selector valve, one of the at least two input ports is coupled to the first port, and the other of the at least two ports is coupled to the third port.

Aspect 9 provides a cleaning apparatus of Aspects 3-8, wherein the control unit includes:
a first timer for activating the first pump; and
a second timer for activating the second pump.

Aspect 10 provides a cleaning apparatus of Aspects 3-9, wherein said first and second pumps are selected from one of a rotary vane pump and a bilge pump.

Aspect 11 provides a cleaning apparatus of Aspects 1-10, wherein the spray nozzle is rotatable.

Aspect 12 provides a cleaning apparatus of Aspects 1-11, wherein the power source includes a rechargeable battery.

Aspect 13 provides a cleaning apparatus of Aspects 1-12, wherein the cleaning chemistry includes a descaler and neutralizing agent.

Aspect 14 provides a cleaning apparatus of Aspects 1-13, wherein the cleaning chemistry includes an acid-based cleaning agent.

Aspect 15 provides a cleaning apparatus of Aspects 1-14, wherein the reservoir is at least a portion of an interior chamber of a housing that houses the first and second pumps, the power source, and the control unit.

Aspect 16 provides a cleaning apparatus of Aspects 1-15, further comprising a housing having an interior to house the fluid transport system, the reservoir, the power source, and the control unit.

Aspect 17 provides a cleaning apparatus of Aspects 1-16, wherein the control unit includes a wireless remote control.

Aspect 18 provides a method of cleaning a chamber of a steam sterilizer using a cleaning apparatus, said method comprising:
opening the door of a steam sterilizer to gain access to the sterilizer chamber;
locating the cleaning apparatus within a chamber of the steam sterilizer, said cleaning apparatus adapted to spray a cleaning chemistry inside the chamber;
initiating operation of the cleaning apparatus to commence a cleaning cycle;
closing the door of the steam sterilizer to enclose the cleaning apparatus within the sterilizer chamber;
reopening the door after completion of the cleaning cycle; and
removing the cleaning apparatus from the chamber of the steam sterilizer.

Aspect 19 provides a method of Aspect 18, wherein operation of the cleaning cycle is initiated by use of a start switch.

Aspect 20 provides a method of Aspects 18-19, wherein operation of the cleaning cycle is initiated by a wireless remote control.

Aspect 21 provides a method of Aspects 18-20, wherein said cleaning apparatus is fully self-contained within the sterilizer chamber.

Aspect 22 provides a method of Aspects 18-21, wherein initiating operation of the cleaning apparatus includes creating a suction within a housing of the cleaning apparatus.

Aspect 23 provides a method for simultaneously cleaning a plurality of steam sterilizer chambers using a plurality of cleaning apparatus, said method comprising:
opening the doors of a plurality of steam sterilizers to gain access to each of respective sterilizer chambers;
locating a cleaning apparatus within each sterilizer chamber of the steam sterilizers, said cleaning apparatus adapted to spray a cleaning chemistry inside the sterilizer chamber;
initiating operation of each of the cleaning apparatus to commence a cleaning cycle;
closing the door of each steam sterilizer to enclose each cleaning apparatus within each of the sterilizer chambers;
reopening the doors of each steam sterilizer after completion of the cleaning cycle; and
removing each of the cleaning apparatus from the sterilizer chambers.

Aspect 24 provides a method of Aspect 23, wherein operation of the cleaning cycle for at least one of the cleaning apparatus is initiated by use of a start switch.

Aspect 25 provides a method of Aspects 23-24, wherein operation of the cleaning cycle is initiated for at least one of the cleaning apparatus by use of a wireless remote control.

Aspect 26 provides a method of Aspects 23-25, wherein each of said cleaning apparatus are respectively fully self-contained within the sterilizer chambers.

Aspect 27 provides a method of Aspect 23-26, wherein initiating operation of each cleaning apparatus includes creating suction within a housing of each cleaning apparatus.

The foregoing describes specific embodiment of the present invention. It should be appreciated that these embodiments are described for purposes of illustration only, and that numerous alterations and modifications may be practiced by those skilled in the art without departing from the spirit and scope of the invention. It is intended that all such modifications and alterations be included insofar as they come within the scope of the invention as claimed or the equivalents thereof.

What is claimed is:

1. A cleaning apparatus for cleaning a chamber, comprising:
a reservoir for storing a cleaning chemistry;
a spray nozzle for distributing the cleaning chemistry within the chamber:
a fluid transport system for conveying the cleaning chemistry from the reservoir to the spray nozzle and returning the cleaning chemistry from the chamber to a reservoir;
a power source for powering the pumping system; and
a control unit for controlling operation of the pumping system.

2. The cleaning apparatus according to claim 1, wherein the fluid transport system is comprised of:
a first pump for conveying the cleaning chemistry from the reservoir to the spray nozzle; and
a second pump for returning the cleaning chemistry from the chamber to the reservoir.

3. The cleaning apparatus according to claim 2, wherein the reservoir comprises a floor and a first port disposed in the floor, and the second pump comprises a suction port fluidically coupled to the first port.

4. The cleaning apparatus according to claim 2, wherein the reservoir comprises a floor and at least one side wall, further comprising:
a second port disposed in the at least one side wall; and
a selector valve having an output port and at least two input ports, the selector valve configured to selectively couple one of the at least two input ports to the output port, wherein the second pump is coupled to the output port of the selector valve, one of the at least two input ports is coupled to the first port, and the other of the at least two ports is coupled to the second port.

5. The cleaning apparatus according to claim 2, further comprising a housing having an interior chamber, wherein the second pump is configured to create a suction that draws fluid into the interior chamber.

6. The cleaning apparatus according to claim 2, wherein the reservoir comprises a floor and at least one side wall, further comprising a first port disposed in the floor of the reservoir and a second port disposed in the at least one side wall of the reservoir, wherein the second pump comprises a suction port fluidically coupled to the first port and an exhaust port fluidically coupled to the second port.

7. The cleaning apparatus according to claim 2, further comprising:
a third port disposed in the at least one side wall; and
a selector valve having an output port and at least two input ports, the selector valve configured to selectively couple one of the at least two input ports to the output port,
wherein the suction port of the second pump is coupled to the output port of the selector valve, one of the at least two input ports is coupled to the first port, and the other of the at least two ports is coupled to the third port.

8. The cleaning apparatus according to claim 2, wherein the control unit includes:
a first timer for activating the first pump; and
a second timer for activating the second pump.

9. The cleaning apparatus according to claim 2 wherein said first and second pumps are selected from one of a rotary vane pump and a bilge pump.

10. The cleaning apparatus according to claim 1, wherein the spray nozzle is rotatable.

11. The cleaning apparatus according to claim 1, wherein the power source includes a rechargeable battery.

12. The cleaning apparatus according to claim 1, wherein the cleaning chemistry includes a descaler and neutralizing agent.

13. The cleaning apparatus according to claim 1, wherein the cleaning chemistry includes an acid-based cleaning agent.

14. The cleaning apparatus according to claim 1, wherein the reservoir is at least a portion of an interior chamber of a housing that houses the first and second pumps, the power source, and the control unit.

15. The cleaning apparatus according to claim 1, further comprising a housing having an interior to house the fluid transport system, the reservoir, the power source, and the control unit.

16. The cleaning apparatus according to claim 1, wherein the control unit includes a wireless remote control.

17. A method of cleaning a chamber of a steam sterilizer using a cleaning apparatus, said method comprising:
opening the door of a steam sterilizer to gain access to the sterilizer chamber;
locating the cleaning apparatus within a chamber of the steam sterilizer, said cleaning apparatus adapted to spray a cleaning chemistry inside the chamber;
initiating operation of the cleaning apparatus to commence a cleaning cycle;
closing the door of the steam sterilizer to enclose the cleaning apparatus within the sterilizer chamber;
reopening the door after completion of the cleaning cycle; and
removing the cleaning apparatus from the chamber of the steam sterilizer.

18. The method according to claim 17, wherein operation of the cleaning cycle is initiated by a wireless remote control.

19. The method according to claim 17, wherein said cleaning apparatus is fully self-contained within the sterilizer chamber.

20. The method according to claim 17, wherein initiating operation of the cleaning apparatus includes creating a suction within a housing of the cleaning apparatus.
